(19) United States
(12) Patent Application Publication
(10) Pub. No.: US 2019/0125420 A1
(43) Pub. Date: May 2, 2019

(54) Bioabsorbable Composite Screw

(71) Applicant: Bone Solutions, Inc., Colleyville, TX (US)

(72) Inventors: Drew Diaz, Colleyville, TX (US); Frankie L. Morris, Colleyville, TX (US)

(21) Appl. No.: 16/176,558

(22) Filed: Oct. 31, 2018

Related U.S. Application Data
(60) Provisional application No. 62/579,223, filed on Oct. 31, 2017.

(51) Int. Cl. A61B 17/86; A61F 2/08; A61L 31/12; A61L 31/14; A61L 31/16

(52) U.S. Cl. CPC A61B 17/866; A61B 17/8615; A61B 17/8625; A61B 17/8635; A61F 2/0811; A61B 2017/00004; A61B 17/8685; A61L 31/128; A61L 31/146; A61L 31/148; A61L 31/16; A61B 17/864

(57) ABSTRACT

The present disclosure provides a bioabsorbable composite screw and methods of use. The bioabsorbable composite screw includes an elongated body having a proximal end and a distal end. The elongated body includes an outer surface provided with a plurality of threads defined by a plurality of crests and a plurality of roots. The bioabsorbable composite screw also includes a drive socket positioned at the proximal end of the elongated body. The bioabsorbable composite screw including both the elongated body and the drive socket is made from a polymer including poly-lactic acid and either magnesium phosphate or potassium phosphate.

### RELATED APPLICATIONS

This application claims priority to U.S. Provisional Application No. 62/579,223 entitled “Bioabsorbable Composite Screw,” filed on Oct. 31, 2017, the contents of which are hereby incorporated by reference in its entirety.

### FIELD OF THE INVENTION

The invention relates to the field of bioabsorbable composite screws, and particularly those used for fixing orthopedic implants to or in a bone of a patient.

### BACKGROUND OF THE INVENTION

Unless otherwise indicated herein, the materials described in this section are not prior art to the claims in this application and are not admitted to be prior art by inclusion in this section.

Many procedures in the field of orthopedics require the use of screws. In one example, screws may be used to attach soft tissue such as ligaments, tendons, or muscles to a surface from which the soft tissue has become detached. For example, the rotator cuff may be reattached to the humeral head during a shoulder repair. As another example, screws may be used in the reconstruction of the anterior cruciate ligament (ACL) to secure a substitute ligament to the tibia and the femur. Screws may also be used to secure soft tissue to supplementary attachment sites for reinforcement. For example, in urological applications, screws may be used in bladder neck suspension procedures to attach a portion of the bladder to an adjacent bone surface. Such soft tissue attachments may be done during either open or closed surgical procedures, the latter being generally referred to as arthroscopic or endoscopic surgery. The terms “arthroscopic” and “endoscopic” may be used interchangeably herein and are intended to encompass arthroscopic, endoscopic, laparoscopic, hysteroscopic or any other similar surgical procedures performed with elongated instruments inserted through small openings in the body. Many other potential uses of biocompatible screws are possible as well.

Such screws make take a variety of forms. Typically, metal screws (often made of titanium) are used for this purpose and are the preferred choice for surgeons as they cause minor inflammatory response. However, the use of metal screws often requires a second surgical intervention to remove the screw after healing. Also, because mechanical stresses are borne to a large part by rigid metal screws, the surrounding bone does not carry sufficient load during and after the healing process to produce a biologically strong structure. In some cases, this can cause rise to post-operative complications a number of years after implantation.

Synthetic polymer screws are currently available and are an alternative choice to metal screws. As the polymer is degraded and absorbed by the body during the months following surgery, the screw site is replaced by biological tissue and so the biomechanical stresses are transferred from the implant or screw to the newly-formed tissue produced during the healing process. Such synthetic polymer screws are typically heavy in poly-lactic acid and tri-calcium phosphate, the combination of which take a long time to be absorbed into the body. The use of different types of materials such as magnesium-based, iron-based, and zinc-based alloys may help to speed up absorption rates with their known biocompatibility and material properties. The problem with such materials is that in their purist form, these materials have shown to have high corrosion effects during in-vitro and in vivo experiments.

### SUMMARY OF THE INVENTION

In view of the foregoing, the inventors recognized that an improved bioabsorbable composite screw would be desirable. The present invention provides such a device and method of use.

Thus, in a first aspect, the present invention provides a bioabsorbable composite screw comprising an elongated body having a proximal end and a distal end, wherein the elongated body includes an outer surface provided with a plurality of threads defined by a plurality of crests and a plurality of roots. The bioabsorbable composite screw also comprises a drive socket positioned at the proximal end of the elongated body. The bioabsorbable composite screw including both the elongated body and the drive socket comprises a polymer including poly-lactic acid and either magnesium phosphate or potassium phosphate.

In a second aspect, the present invention provides for securing a bioabsorbable composite screw to a bone, the method comprising: (a) providing the bioabsorbable composite screw, the bioabsorbable composite screw comprising an elongated body having a proximal end and a distal end, wherein the elongated body includes an outer surface provided with a plurality of threads, and wherein the bioabsorbable composite screw comprises a polymer including poly-lactic acid and either magnesium phosphate or potassium phosphate, (b) inserting the distal end of the elongated body of the bioabsorbable composite screw into a tunnel in the bone, and (c) rotating the elongated body into the tunnel.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a side view of a bioabsorbable composite screw according to an exemplary embodiment of the invention.

FIG. 2 illustrates a perspective view of the bioabsorbable composite screw of FIG. 1 according to an exemplary embodiment of the invention.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The exemplary embodiments described herein are not meant to be limiting. It will be readily understood that the aspects of the present disclosure, as generally described herein and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

As used herein, with respect to measurements, “about” means+/−5%.

As used herein, “osteostimulative” refers to the ability of a material to improve healing of bone injuries or defects.

As used herein, “osteoconductive” refers to the ability of a material to serve as a scaffold for viable bone growth and healing.

As used herein, “osteoinductive” refers to the capacity of a material to stimulate or induce bone growth.

As used herein, “biocompatible” refers to a material that elicits no significant undesirable response when inserted into a recipient (e.g., a mammalian, including human, recipient).

As used herein, “bioabsorbable” refers to a material's ability to be absorbed in-vivo through bodily processes. The absorbed material may turn into bone in the patient's body.

The present disclosure provides a bioabsorbable composite screw suitable for use in orthopedic surgery, with a cannulation, to assist biodegradation of the screw. The bioabsorbable screw described herein may be used in conjunction with cement or bone void fillers. The use of a polymer comprising poly-lactic acid and either magnesium phosphate or calcium phosphate further allows the bioabsorbable composite screw to be absorbed in-vivo, producing increased fixation strength and faster absorption into the body.

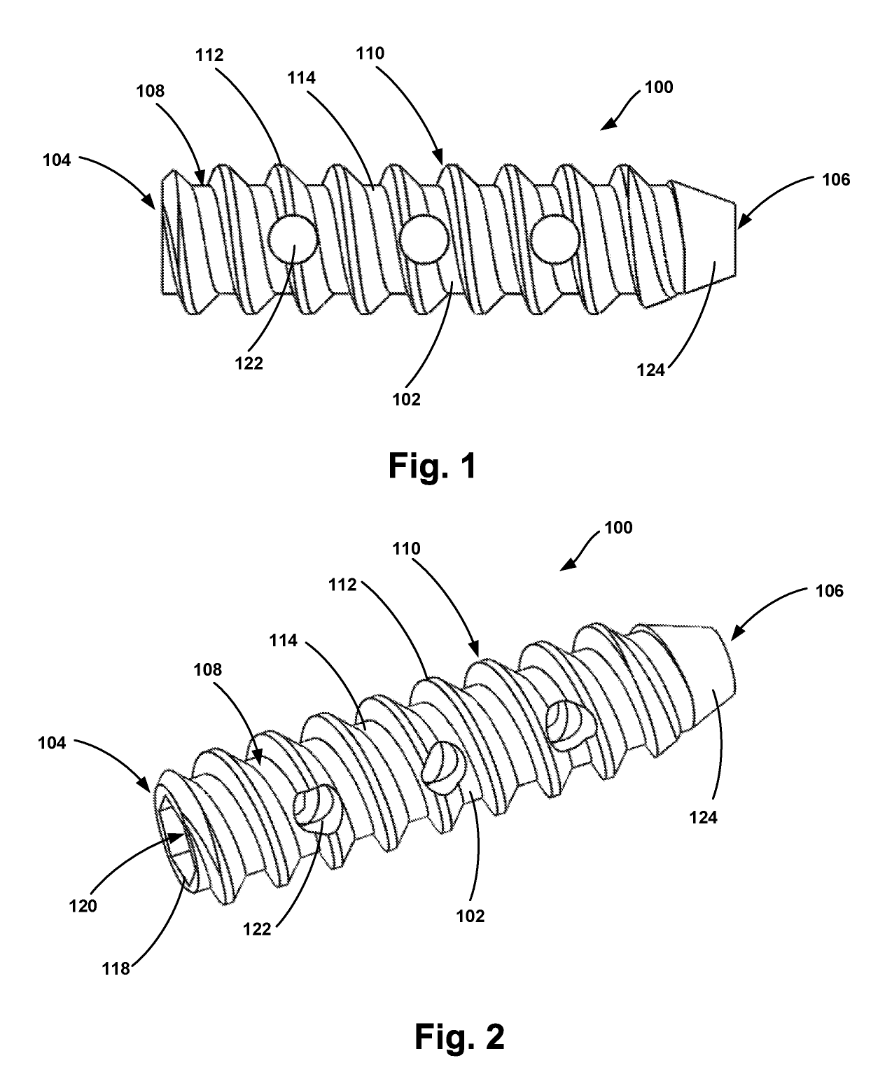

With reference to the Figures, FIGS. 1-2 illustrate an exemplary bioabsorbable composite screw 100. The bioabsorbable composite screw 100 includes an elongated body 102 having a proximal end 104 and a distal end 106. The elongated body 102 includes an outer surface 108 provided with a plurality of threads 110 defined by a plurality of crests 112 and a plurality of roots 114. The bioabsorbable composite screw 100 also includes a drive socket 118 positioned at the proximal end 104 of the elongated body 102. As shown in FIG. 2, the drive socket 118 may comprise a recessed cutout in the proximal end 104 of the elongated body 102. In one example, the bioabsorbable composite screw 100 also includes a head coupled to the proximal end 104 of the elongated body 102. The head may have a diameter greater than a diameter of the elongated body 102, thereby providing a stopping point for the bioabsorbable composite screw 100 as it is inserted into a structure when in use. The elongated body 102 may be integral to the head such that they are formed unitarily, or the elongated body 102 may be coupled separately to the head. In such an example, the drive socket 118 may be integral to the head, such that the drive socket 118 comprises a recessed cutout in the head.

The drive socket 118 is designed to interact with a suitable torque-transmitting insertion device, such as an implant driver, and thereby allow transmission of the requisite amount of torque needed to drive the implant into the prepared socket. For example, the drive socket 118 may be a polygonal recess in the proximal end 104 of the elongated body 102 while the torque-transmitting feature characterizing the distal end of the driver is a corresponding polygonal protrusion (such as found on the conventional hex key or “Allen” key). In another embodiment, the drive socket 118 may be one or more axially extending slots recessed in the proximal end 104 of the elongated body 102 while the driver is a slotted, flat blade or crosshead (“Phillips head”) screwdriver. However, other embodiments will be readily apparent to the skilled artisan. Moreover, it will be readily understood by the skilled artisan that the position of the respective coordinating elements (e.g., recessed slots and grooves that mate with assorted projecting protrusions, protuberances, tabs and splines) may be exchanged and/or reversed as needed.

The entirety of the bioabsorbable composite screw 100 including each of the elongated body 102 and the drive socket 118 is made from a polymer including poly-lactic acid and either magnesium phosphate or potassium phosphate. As used herein, “poly-lactic acid” or polylactide (PLA) is a biodegradable and bioactive thermoplastic aliphatic polyester derived from renewable resources, and may take a variety of forms including, but not limited to, poly-L-lactide (PLLA), poly-D-lactide (PDLA), and poly(L-lactide-co-D,L-lactide) (PLDLLA). As used herein, “magnesium phosphate” is a general term for salts of magnesium and phosphate appearing in several forms and several hydrates including, but not limited to, monomagnesium phosphate ((Mg(H₂PO₄)₂).xH₂O), dimagnesium phosphate ((MgHPO₄).xH₂O), and trimagnesium phosphate ((Mg₃(PO₄)₂).xH₂O). As used herein, “calcium phosphate” is a family of materials and minerals containing calcium ions (Ca²⁺) together with inorganic phosphate anions and appearing in a variety of forms including, but not limited to, monocalcium phosphate, dicalcium phosphate, tricalcium phosphate, octacalcium phosphate, amorphous calcium phosphate, dicalcium diphosphate, calcium triphosphate, hydroxyapatite, apatite, and tetracalcium phosphate.

Making the entirety of the bioabsorbable composite screw 100 including each of the elongated body 102 and the drive socket 118 from a polymer including poly-lactic acid and either magnesium phosphate or potassium phosphate has a number of advantages. In particular, the material of the bioabsorbable composite screw 100 allows the bioabsorbable composite screw 100 to be absorbed in-vivo, producing increased fixation strength and faster absorption into the body. In contrast to traditional metal alloy bone screws, there is no need to remove the bioabsorbable composite screw 100 after a certain period of time because the material of the bioabsorbable composite screw 100 enables bone to actually replace the structure of the bioabsorbable composite screw 100. As such, there is no void that is left behind after the bioabsorbable composite screw 100 is absorbed in-vivo. Instead, the bioabsorbable composite screw 100 is replaced with bone structure grown naturally in the body and the resulting fixation strength is very strong.

The plurality of threads 110 of the bioabsorbable composite screw 100 are preferably positioned along substantially the entire length of the elongated body 102 to maximize fixation strength when inserted into a tunnel in a bone. Each of the plurality of threads 110 on the outer surface 108 of the elongated body 102 has a height defined the diameter of a given crest of the plurality of crests 112 (e.g., a major diameter of the thread) less the diameter of a given root of the plurality of roots 114 (e.g., a minor diameter of the thread). Further, each of the plurality of threads 110 has a width defined as the measurement across a given thread at half the height of the given thread. In one embodiment, each of the plurality of threads 110 have substantially the same height. Alternatively, the thread height may taper from the proximal end 104 of the elongated body 102 to distal end 106 of the elongated body 102. A ratio of thread width to thread height may range from about 0.3 to about 2.

In one example, an inner surface of the elongated body includes a central lumen 120 that extends from the proximal end 104 to the distal end 106 of the elongated body 102. The central lumen 120 may be able to receive a k-wire and/or a guide pin when in use. In one example, an inner diameter of the central lumen 120 is stepped such that an inner diameter of the proximal end 104 of the elongated body 102 is greater than an inner diameter of the distal end 106 of the elongated body 102. In another example, the elongated body includes one or more cavities 122 in the outer surface 108 of the elongated body 102. In one example, the one or more cavities 122 connect the central lumen 120 of the elongated body 102 to the outer surface 108 of the elongated body 102. In one particular example, the bioabsorbable composite screw 100 has a multi-part construction that enables the bioabsorbable composite screw 100 to be split apart to give access to the central lumen 120.

In one example, the elongated body 102 tapers from the proximal end 104 to the distal end 106. In another example, the elongated body 102 has a substantially similar diameter from the proximal end 104 to the distal end 106. The bioabsorbable composite screw 100 may also include a tip 124 disposed at the distal end 106 of the elongated body 102. The tip 124 may be non-threaded so as to minimize abrasion with the graft and include a taper for ease of insertion into a tunnel in a bone.

In accordance with a further aspect of the invention, the bioabsorbable composite screw 100 further include a bioactive therapeutic agent for achieving further enhanced bone fusion and ingrowth. In one example, the bioactive therapeutic agent is in the form of a surface coating on the outer surface 108 of the bioabsorbable composite screw 100. In another example, the bioabsorbable composite screw 100 may be incorporated into a plurality of pores in the polymer material from which the bioabsorbable composite screw 100 is formed. Such bioactive therapeutic agents may include natural or synthetic therapeutic agents such as bone morphogenic proteins (BMPs), growth factors, bone marrow aspirate, stem cells, progenitor cells, antibiotics, or other osteoconductive, osteoinductive, osteogenic, bio-active, or any other fusion enhancing material or beneficial therapeutic agent.

The resultant bioabsorbable composite screw 100 exhibits relatively high mechanical strength for load bearing support, while additionally and desirably providing high osteoconductive and osteoinductive properties to achieve enhanced bone ingrowth and fusion. In use, the polymer including poly-lactic acid and either magnesium phosphate or potassium phosphate that makes up the bioabsorbable composite screw 100 will induce bone growth into the bioabsorbable composite screw 100 and be resorbed. The bioabsorbable composite screw 100 is eventually replaced by bone in the body, thereby firmly securing the component to which the bioabsorbable composite screw 100 (e.g., a substitute ligament in an ACL reconstruction surgery or an existing rotator cuff in a rotator cuff reattachment surgery) is connected to the bone structure the body.

In operation, the present invention provides a method for securing a bioabsorbable composite screw to a bone, the method comprising: (a) providing the bioabsorbable composite screw, the bioabsorbable composite screw comprising an elongated body having a proximal end and a distal end, wherein the elongated body includes an outer surface provided with a plurality of threads, and wherein the bioabsorbable composite screw comprises a polymer including poly-lactic acid and either magnesium phosphate or potassium phosphate, (b) inserting the distal end of the elongated body of the bioabsorbable composite screw into a tunnel in the bone, and (c) rotating the elongated body into the tunnel.

Rotating the elongated body into the tunnel may comprise inserting a torque-transmitting insertion device, such as an implant driver, into a drive socket at the proximal end of the elongated body, and rotating the torque-transmitting insertion device.

In one example, an inner surface of the elongated body includes a central lumen that extends from the proximal to the distal end of the elongated body. In such an example, inserting the distal end of the elongated body of the bioabsorbable composite screw into the tunnel in the bone comprises inserting the central lumen of the elongated body over a guide pin.

In another example, the method further includes forming the tunnel in the bone. Such a tunnel may be formed using a drill, for example. In another example, the method further includes inserting a ligament (e.g., a central third of the patellar tendon or a braided hamstring for an ACL reconstruction surgery or an existing rotator cuff in a rotator cuff reattachment surgery) in the tunnel in the bone, and inserting the distal end of the elongated body of the bioabsorbable composite screw into the tunnel in the bone such that said elongated body fills a substantial portion of the tunnel, wherein at least some of the plurality of threads engage the bone in an inner surface of the tunnel, and wherein the ligament is securely fixed between the at least some of the plurality of threads of the elongated body and the inner surface of the tunnel in the bone.

In another example, an inner surface of the elongated body includes a central lumen that extends from the proximal to the distal end of the elongated body. In such an example, the method further includes applying an uncured osteostimulative material to the central lumen of the elongated body. In yet another example, the elongated body includes one or more cavities located in the outer surface of the elongated body, wherein the one or more cavities connect connecting the central lumen of the elongated body to an outer surface of the elongated body. In such an example, the method further includes applying an uncured osteostimulative material to the central lumen of the elongated body and/or the one or more cavities of the elongated body.

The osteostimulative material may take a variety of forms. The osteostimulative material may allow for in-situ (i.e., in vivo) attachment of biological structures to each other and to manmade structures. The osteostimulative material may also facilitate the repair of bone, ligaments, tendons and adjacent structures. The osteostimulative material may also provide a bone substitute for surgical repair. The formulation of the osteostimulative material is usable at numerous temperatures, pH ranges, humidity levels, and pressures. However, the formulation can be designed to be utilized at all physiological temperatures, pH ranges, and fluid concentrations. The osteostimulative material typically is, but not necessarily, injectable before curing and can exhibit neutral pH after setting. It may be absorbed by the host over a period of time.

Generally, the osteostimulative material is derived from the hydrated mixture which comprises: (a) KH₂PO₄ in an amount between about 20-70 dry weight percent, (b) MgO in an amount between 10-50 dry weight percent, (c) a calcium containing compound, and (d) a sugar. In one particular example, the calcium containing compound is Ca₅(PO₄)₃OH.

Exemplary formulations of the osteostimulative material include the following:

Formulation I*

| | |
|---|---|
| Potassium phosphate (i.e., KH₂PO₄) | 61% |
| MgO (calcined) | 31% |
| Ca₁₀(PO₄)₆(OH)₂ | 4% |
| Sucrose C₁₂H₂₂O₁₁(powder) | 4% |

*All values are weight percentages

Water is added up to about 40 weight percent of the dry formulation, preferably between about 20-35 weight percent, more preferably between 22-25 weight percent.

Formulation II*

| | |
|---|---|
| KH₂PO₄ | 54% |
| MgO (calcined) | 33% |
| Ca₁₀(PO₄)₆(OH)₂ | 9% |
| Sucrose C₁₂H₂₂O₁₁(powder) | 4% |

*All values are weight percentages

Water is added up to about 40 weight percent of the dry formulation, preferably between about 20-35 weight percent, more preferably between 22-25 weight percent.

Formulation III*

| | |
|---|---|
| KH₂PO₄ | 44% |
| MgO (calcined) | 44% |
| Calcium-containing compound | 8% (whereby the compound is |
| | Ca₁₀(PO₄)₆(OH)₂ or CaSiO₃) |
| Sucrose C₁₂H₂₂O₁₁(powder) | 4% |

*All values are weight percentages

Water is added up to about 40 weight percent of the dry formulation, preferably between about 20-35 weight percent, more preferably between 22-25 weight percent.

Formulation IV*

| | |
|---|---|
| KH₂PO₄ | 44% |
| MgO (calcined) | 41% |
| Ca₁₀(PO₄)₆(OH)₂ | 8% |
| Sucrose C₁₂H₂₂O₁₁(powder) | 4% |
| Mono-sodium phosphate (MSP) | 3% |

*All values are weight percentages

Water is added up to about 40 weight percent of the dry formulation, preferably between about 20-35 weight percent, more preferably between about 28-32 weight percent.

Formulation V*

| | |
|---|---|
| Potassium phosphate (i.e., KH₂PO₄) | 41% |
| MgO (calcined) | 45% |
| Calcium-containing compound | 9% (whereby the compound is |
| | Ca₁₀(PO₄)₆(OH)₂, CaSiO₃ or |
| | combinations thereof.) |
| Sucrose C₁₂H₂₂O₁₁(powder) | 1% |

*All values are weight percentages

Water is added up to about 40 weight percent of the dry formulation, preferably between about 20-35 weight percent, more preferably between 22-25 weight percent.

Formulation VI*

| | |
|---|---|
| KH₂PO₄ | 45% |
| MgO (calcined) | 45% |
| Ca₁₀(PO₄)₆(OH)₂ | 8% |
| Sucralose | 2% |

*All values are weight percentages

Water is added up to about 40 weight percent of the dry formulation, preferably between about 20-35 weight percent, more preferably between 22-25 weight percent.

Formulation VII*

| | |
|---|---|
| KH₂PO₄ | 61% |
| MgO (calcined) | 32% |
| Ca₁₀(PO₄)₆(OH)₂ | 4% |
| Dextrose | 1.5% |
| α-Ca₃(PO₄)₂ | 1.5% |

*All values are weight percentages

Water is added up to about 40 weight percent of the dry formulation, preferably between about 20-35 weight percent, more preferably between 22-25 weight percent.

Formulation VIII*

| | |
|---|---|
| KH₂PO₄ | 50% |
| MgO (calcined) | 35% |
| Ca₁₀(PO₄)₆(OH)₂ | 7% |
| β-Ca₃(PO₄)₂ | 3% |
| Dextrose | 5% |

*All values are weight percentages

Water is added up to about 40 weight percent of the dry formulation, preferably between about 20-35 weight percent, more preferably between 22-25 weight percent.

Formulation IX*

| | |
|---|---|
| KH₂PO₄ | 54% |
| Phosphoric Acid | 4% |
| Metal oxide | 32% (wherein the metal oxide is MgO, |
| | ZrO, FeO or a combination thereof) |
| Ca₁₀(PO₄)₆(OH)₂ | 7% |
| Sucrose | 3% |

*All values are weight percentages

Water is added up to about 40 weight percent of the dry formulation, preferably between about 20-35 weight percent, more preferably between 22-25 weight percent.

Formulation X*

| | |
|---|---|
| KH₂PO₄ | 61% |
| Metal oxide | 32% (wherein the metal oxide is MgO, |
| | Ca, FeO or a combination thereof) |
| Ca₁₀(PO₄)₆(OH)₂ | 6% |
| Sucrose | 1% |

*All values are weight percentages

Water is added up to about 40 weight percent of the dry formulation, preferably between about 20-35 weight percent, more preferably between 22-25 weight percent.

Formulation XI*

| | |
|---|---|
| KH₂PO₄ | 45% |
| MgO (calcined) | 45% |
| Ca₁₀(PO₄)₆(OH)₂ | 10% |

*All values are weight percentages

Water is added up to about 40 weight percent of the dry formulation, preferably between about 20-35 weight percent, more preferably between 22-25 weight percent.

The above formulations and weight percentages are merely exemplary. A range of dry constituents can also be used. For example, a suitable range for the phosphate (i.e., mono-potassium phosphate (MKP)) is generally between about 20-70 weight percent, preferably between about 40-65 weight percent. In some situations and/or embodiments it is preferable to use the phosphate at a range between about 40-50 weight percent, while in others it may be preferable to use a range of about 50-65 weight percent.

A suitable range for the metal oxide (i.e., MgO) is generally between about 10-60, preferably between 10-50, and even more preferably between 30-50 weight percent. In some situations and/or embodiments it may be preferable to use between about 35 and 50 weight percent.

Calcium containing compounds can be added in various weight percentages. The calcium containing compound(s) is preferably added at about 1-15 weight percent, more preferably between about 1-10 weight percent. Higher percentages can be employed in certain situations.

Sugars (and/or other carbohydrate containing substances) are generally present at weight percent between 0.5 and 20, preferably about 0.5-10 weight percent of the dry composition.

Water (or another aqueous solution) can be added in a large range of weight percentages generally ranging from about 15-40 weight percent, preferably between about 20-35 weight percent. For example, in certain embodiments of the materials as generally described herein, water or other aqueous solution is added at between about 28-32 weight percent. In other embodiments of the materials as generally described herein, water or other aqueous solution is added at between about 28-32 weight percent. It was found that a saline solution may be used. An exemplary saline solution is a 0.9% saline solution.

For some embodiments (i.e., formula III) it has been found that adding water at a weight percent of about 37 weight percent produces a creamy textured material that is extremely easy to work with, has excellent adhesive properties, and is easily injectable through a syringe.

The noted ranges may vary with the addition of various fillers and other components or for other reasons.

In one embodiment, the weight percent ratio between MKP and MgO is between about 4:1 and 0.5:1. In another it is between approximately 2:1 and 1:1.

Without limiting the invention in any manner, in such an embodiment the inventors surmise that the un-reacted magnesium is at least partly responsible for the in vivo expandability characteristics of the bio-adhesive. Specifically the metal oxide (i.e., magnesium oxide) reacts with water and serum in and around the living tissue to yield Mg(OH)₂ and magnesium salts. It has been found that in some embodiments the material generally expands to between 0.15 and 0.20 percent of volume during curing in moisture. The expansion of the material is believed to increase the adhesive characteristics of the material. For example, the disclosed material has been shown to effectively attach soft tissues like ligaments to bone, the expansion of the material improving adhesion through mechanical strength.

Osteostimulative material useful in the present invention can also be found in U.S. Pat. Nos. 6,533,821, 6,787,495, 7,045,476, 9,078,884, U.S. Patent Application Publication No. 2015/0250924, and U.S. Patent Application Publication No. 2015/0314045, all of which are hereby incorporated by reference in their entirety.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Because many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

What is claimed is:

1. A bioabsorbable composite screw, comprising:
an elongated body having a proximal end and a distal end, wherein the elongated body includes an outer surface provided with a plurality of threads defined by a plurality of crests and a plurality of roots; and
a drive socket positioned at the proximal end of the elongated body,
wherein the bioabsorbable composite screw including both the elongated body and the drive socket comprises a polymer including poly-lactic acid and either magnesium phosphate or potassium phosphate.

2. The bioabsorbable composite screw of claim 1, further comprising:
a head coupled to the proximal end of the elongated body, wherein the drive socket is disposed within the head.

3. The bioabsorbable composite screw of claim 2, wherein the elongated body is integral to the head.

4. The bioabsorbable composite screw of claim 1, wherein the drive socket comprises a polygonal recess in the proximal end of the elongated body configured to receive a corresponding polygonal protrusion.

5. The bioabsorbable composite screw of claim 1, wherein the drive socket comprises a plurality of radially extending slots recessed in the proximal end of the elongated body configured to receive corresponding radially extending protrusions.

6. The bioabsorbable composite screw of claim 1, wherein an inner surface of the elongated body includes a central lumen that extends from the proximal end to the distal end of the elongated body.

7. The bioabsorbable composite screw of claim 6, wherein an inner diameter of the central lumen is stepped such that an inner diameter of the proximal end of the elongated body is greater than an inner diameter of the distal end of the elongated body.

8. The bioabsorbable composite screw of claim 1, wherein the elongated body includes one or more cavities located in the outer surface of the elongated body.

9. The bioabsorbable composite screw of claim 1, further comprising:
a tip disposed at the distal end of the elongated body, wherein the tip is non-threaded and includes a taper.

10. The bioabsorbable composite screw of claim 1, wherein the bioabsorbable composite screw has a multi-part construction that enables the bioabsorbable composite screw to be split apart to give access to a central lumen that extends from the proximal end to the distal end of the elongated body.

11. The bioabsorbable composite screw of claim 1, wherein the elongated body tapers from the proximal end to the distal end.

12. The bioabsorbable composite screw of claim 1, wherein the bioabsorbable composite screw further includes a bioactive therapeutic agent.

13. The bioabsorbable composite screw of claim 12, wherein the bioactive therapeutic agent comprises a coating on the outer surface of the elongated body.

14. The bioabsorbable composite screw of claim 12, wherein the bioactive therapeutic agent is incorporated into a plurality of pores in the polymer from which the bioabsorbable composite screw is formed.

15. A method for securing a bioabsorbable composite screw to a bone, the method comprising:
providing the bioabsorbable composite screw, the bioabsorbable composite screw comprising an elongated body having a proximal end and a distal end, wherein the elongated body includes an outer surface provided with a plurality of threads, and wherein the bioabsorbable composite screw comprises a polymer including poly-lactic acid and either magnesium phosphate or potassium phosphate;
inserting the distal end of the elongated body of the bioabsorbable composite screw into a tunnel in the bone; and
rotating the elongated body into the tunnel.

16. The method of claim 15, wherein an inner surface of the elongated body includes a central lumen that extends from the proximal end to the distal end of the elongated body, the method further comprising:
applying an uncured osteostimulative material to the central lumen of the elongated body.

17. The method of claim 16, wherein the elongated body includes one or more cavities located in the outer surface of the elongated body, wherein the one or more cavities connect the central lumen of the elongated body to the outer surface of the elongated body, the method further comprising:
applying an uncured osteostimulative material to the central lumen of the elongated body and/or the one or more cavities of the elongated body.

18. The method of claim 15, wherein an inner surface of the elongated body includes a central lumen that extends from the proximal end to the distal end of the elongated body, and wherein inserting the distal end of the elongated body of the bioabsorbable composite screw into the tunnel in the bone comprises inserting the central lumen of the elongated body over a guide pin.

19. The method of claim 15, further comprising:
forming the tunnel in the bone.

20. The method of claim 15, further comprising:
inserting a ligament in the tunnel in the bone; and
inserting the distal end of the elongated body of the bioabsorbable composite screw into the tunnel in the bone such that said elongated body fills a substantial portion of the tunnel, wherein at least some of the plurality of threads engage the bone in an inner surface of the tunnel, and wherein the ligament is securely fixed between the at least some of the plurality of threads of the elongated body and the inner surface of the tunnel in the bone.
